(19) United States
(12) Plant Patent Application Publication
(10) Pub. No.: US 2026/0271808 P1
(43) Pub. Date: Sep. 10, 2026

(54) Strawberry Plant Named 'CBC019'

(50) ;

(71) Applicant: Kyle M VandenLangenberg, Tracy, CA (US)

(72) Inventor: Kyle M VandenLangenberg, Tracy, CA (US)

(73) Assignee: California Berry Cultivars, LLC, French Camp, CA (US)

(21) Appl. No.: 19/554,511

(22) Filed: Mar. 2, 2026

Related U.S. Application Data
(60) Provisional application No. 63/766,609, filed on Mar. 4, 2025.

(51) Int. Cl. A01H 6/74; A01H 5/08

(52) U.S. Cl. CPC A01H 6/7409; A01H 5/08
USPC PLT/208

(57) ABSTRACT

A new and distinct cultivar of strawberry plant (Fragaria x ananassa) named ‘CBC019’ is presented here. This new day-neutral strawberry cultivar is characterized by moderate vigor plants which produce high yields of large, firm, flavorful fruit throughout the growing season. In addition, plants of ‘CBC019’ are resistant to Macrophomina phaseolina, the causal agent of charcoal rot, and Fusarium oxysporum f. sp fragariae, the causal agent of fusarium wilt in strawberry.

Latin name of the genus and species of the plant claimed: Botanical classification: Fragaria x ananassa.

Variety denomination The new strawberry variety denomination is ‘CBC019’.

### BACKGROUND OF THE INVENTION

This invention relates to a new and distinctive day-neutral type cultivar designated as ‘CBC019’. The new cultivar was the result of a controlled cross performed in 2017 in Huelva, Spain between the cultivar ‘Monterey’ (U.S. Plant Pat. No. 19,767) and the cultivar ‘Cabrillo’ (U.S. Plant Pat. No. 27,830). ‘CBC019’ was first fruited at an experimental orchard near French Camp, California in 2018, where it was selected, originally designated 117.054-106, and propagated asexually by runners. Following selection and during testing, the plant of this selection was later designated ‘CBC019’. Asexual propagules from this original source have been tested in Watsonville, California, in Oxnard, Calif., and to a limited extent in grower fields starting in 2023. The cultivar is stable and reproduces true to type in successive generations of asexual reproduction.

### Comparison with Parents

The parent ‘Monterey’ is a day-neutral cultivar that was selected for its day-neutral flowering habit, good flavor, and high yields. Plants of ‘Monterey’ are larger than ‘CBC019’.The parent ‘Cabrillo’ is a day-neutral cultivar that was selected for its day-neutral flowering habit, high yield, and firm fruit. Plants of ‘Cabrillo’ are similar in size to ‘CBC019’.

### SUMMARY OF THE INVENTION

‘CBC019’ is a day-neutral cultivar that produces fruit over an extended period when treated appropriately in arid, sub-tropical climates. When treated with appropriate planting regimes, ‘CBC019’ has larger fruit and produces individual plant yields greater than that of either parent. The following traits have been repeatedly observed over multiple testing years and locations and are determined to be unique characteristics of ‘CBC019’. These characteristics in combination distinguish ‘CBC019’ as a new and distinct Strawberry plant:
a. Medium sized plant.
b. Day-neutral flowering habit.
c. Medium glossy red fruit.
d. Fruit is firm throughout the growing season.
e. Fruit postharvest longevity is good.
f. Excellent disease resistance.

The cultivar ‘CBC019’, when planted under appropriate conditions, is a continuously flowering day-neutral type with exceptional total yields. Fruit quality is good with excellent flavor. In addition to its yield performance, ‘CBC019’ is resistant to Macrophomina phaseolina, the causal agent of charcoal rot, and Fusarium oxysporum f. sp fragariae, the causal agent of fusarium wilt in strawberry.

### BRIEF DESCRIPTION OF THE FIGURES

The accompanying color photographs, identified as FIGS. 1 through 5, show the appearance of typical specimens of the new strawberry cultivar designated ‘CBC019’. These Figures depict the colors, as nearly true as can be achieved with conventional photography.

FIG. 1 shows a typical leaf and petiole structure.

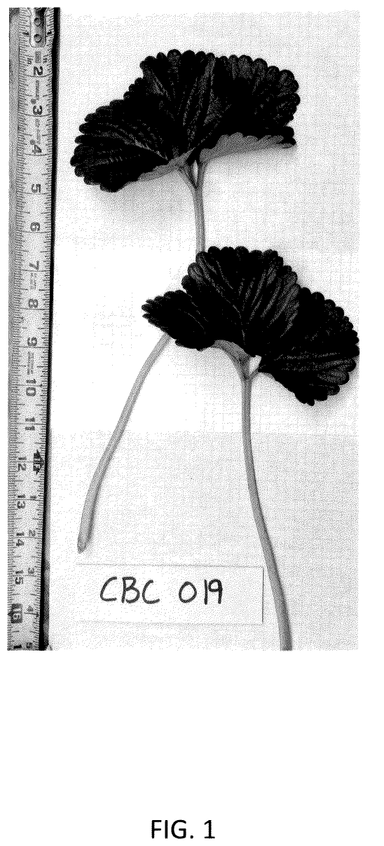

FIG. 2 shows a typical plant and fruit early season.

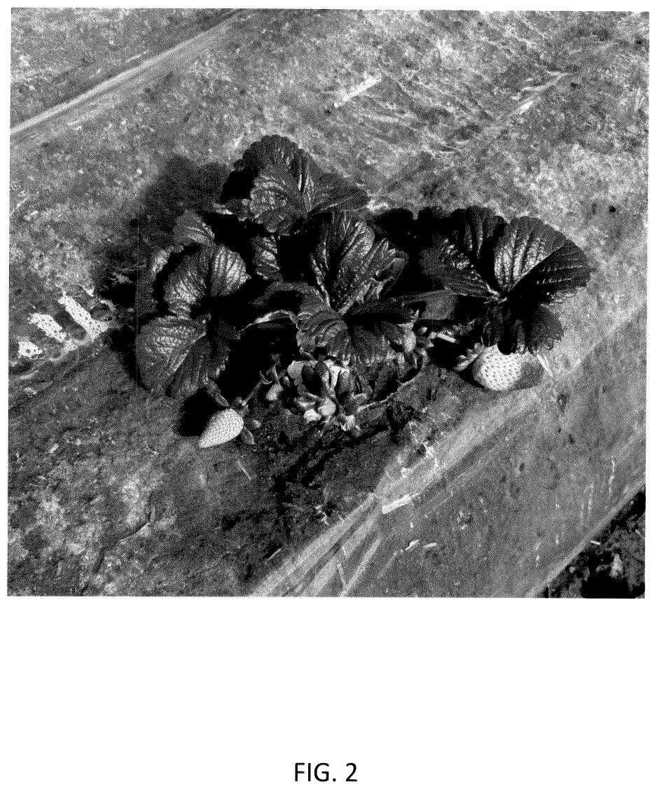

FIG. 3 shows typical fruit in a box.

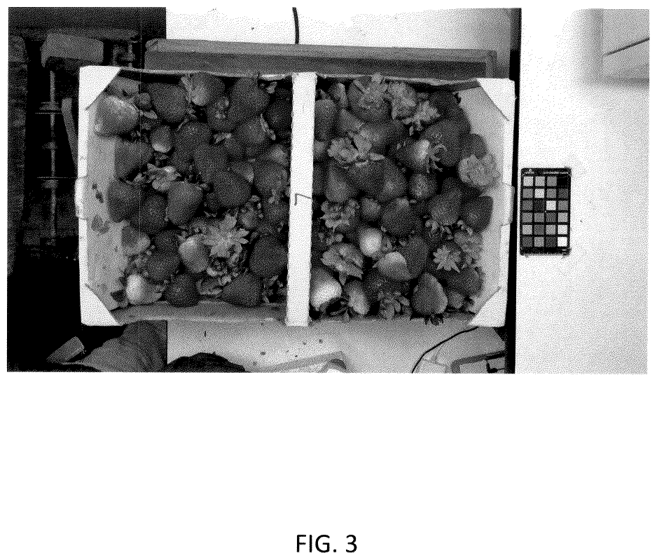

FIG. 4 shows a cross-section of representative fruit mid-season.

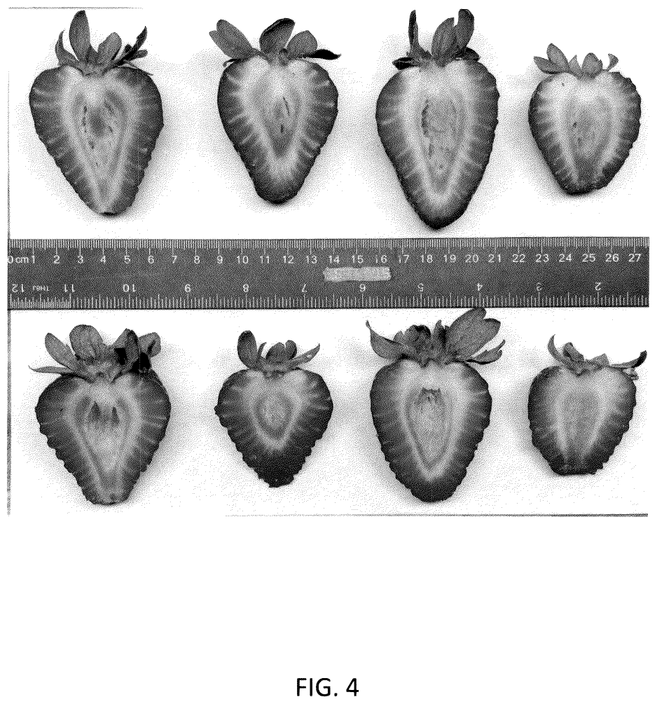

FIG. 5 shows a typical plot in the field of plants early in the season.

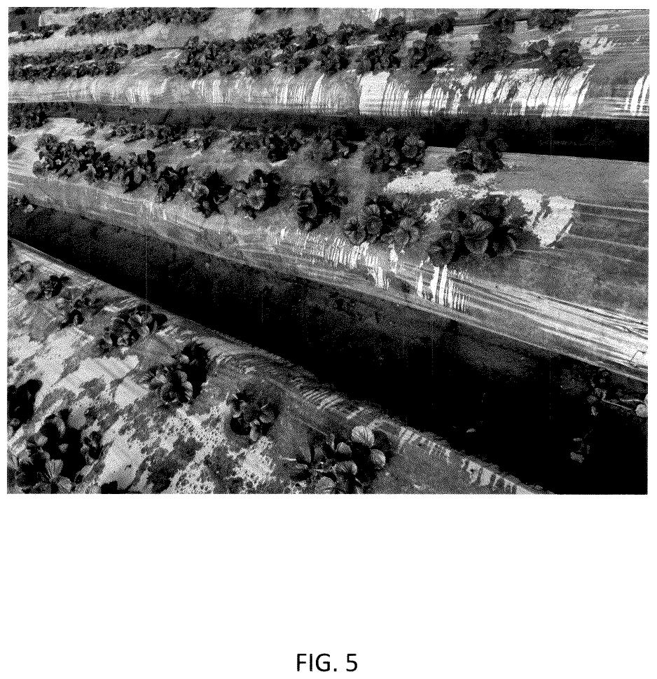

### DETAILED BOTANICAL DESCRIPTION OF THE PLANT

‘CBC019’ is a new and distinct day-neutral cultivar of strawberry, genus, and species Fragaria x ananassa. Plants of ‘CBC019’ bloom continuously in higher elevation plantings as well as the heat of low elevation nursery locations such as French Camp, California, consistent with other day-neutral varieties. The plants of the new cultivar have a moderate vigor.

‘CBC019’ exhibits several characteristics which are improvements over one or both of its parent varieties, and other cultivars known to the Inventor. These characteristics are compared with other cultivars in the following discussion and tables.

The fruit size of ‘CBC019’ is large sized, larger than its parents. The fruit size of ‘CBC019’ is still within an ideal range since it is not too large to be damaged in shipping containers, but not too small to allow for efficient picking and packing. Storability of the fruit has not been measured but in informal observations the fruit retains good physical characteristics and appearance for several days after harvest at room temperature and up to at least a week in common refrigerator conditions of roughly 3° C.

The data set for ‘CBC019’ set forth below was collected in May 2025 from plants grown at a test plot in Watsonville, California. Yield, size, and firmness data is either averaged over the entire 2024-2025 growing season or presented as monthly totals at the same location. Description data was collected on five-month-old plants.

The observed characteristics of ‘CBC019’ are set forth in Table 1, 2, 3, and 4 below. Color terminology, where noted, is in accordance with the Munsell Colorchart. The observed characteristics of ‘CBC019’ are compared to the closest cultivars known to the Inventor, in Table 2, 3, and 4. Table 5 sets forth the test results of DNA fingerprinting using eight SSR markers.

TABLE 1
Detailed Description of Characteristics of 'CBC019'

| | |
|---|---|
| SPECIFICATION: | |
| Genus/Species | Fragaria X ananassa |
| Market Name | Strawberry |
| PARENTS: | |
| Female | ‘Monterey’ (U.S. Plant Pat. No. 19,767) |
| Male | ‘Cabrillo’ (U.S. Plant Pat. No. 27,830) |
| PLANT: | |
| Type | Day-neutral |
| Growth Habit | Upright |
| Foliage density | Moderate |
| Vigor | Moderate |
| Height | Average 28.3 cm; range 25-30 cm |
| Width | Average 35 cm; range 28-43 cm |
| Disease tolerance | Tes t plots have shown tolerance to |
| | Macrophomina phaseolina, the |
| | causal agent of charcoal Fusarium |
| | oxysporum f. sp fragariae, the |
| | agent of fusarium wilt |
| Drought tolerance | Not observed |
| Pest resistance | Not observed |
| LEAF: | |
| Width | 15 cm |
| Color | Adaxial surface: 2.5 G 3/3 |
| | Abaxial surface: 10 GY 7/8 |
| Pubescence | Slight |
| Glossiness | Medium |
| Variegation | Not present |
| TERMINAL LEAFLET | |
| Length | Average 71.4 mm; range 66-78 mm |
| Width | Average 75 mm; range 68-84 mm |
| Ratio (length/width) | 0.95 |
| Margins | Crenate |
| Leaflet shape | Obovate |
| Base shape | Cuneate |
| Cross-section shape | Moderately concave |
| PETIOLE | |
| Pubescence | Slight |
| Petiole color | 5 GY 8/8 |
| Petiole length | Average 14.2 cm; range 12-19 cm |
| Petiole diameter | Average 4.8 mm; range 4-6 mm |
| STOLON | |
| Anthocyanin coloration | Present, medium |
| Pubescence | medium density |
| Diameter | Average 3.7 mm; range 3-4 mm |
| STIPULE | |
| Anthocyanin coloration | Present, light |
| Length | Average 18 mm; range 16-21 mm |
| Width | Average 7 mm; range 6-8 mm |
| INFLORESCENCE: | |
| Flowering | Early to normal |
| Position | Above foliage |
| Petal arrangement | Broadly rounded; uniform |
| Calyx size | Large |
| Number of blooms | 24 |
| Length | 3.3 cm |
| Flower diameter | Average 26 mm; range 23-29 mm |
| Petal number | Average 6; range 5-7 |
| Petal length | Average 9 mm; range 8-10 mm |
| Petal width | Average 10 mm; range 9-11 mm |
| Ratio (length/width) | 0.9 |
| Petal color | N 9.5/0 |
| Calyx color | Adaxial surface: 7.5 GY 5/7 |
| | Abaxial surface: 7.5 GY 8/7 |
| Stamens length | Average 23 mm; range 21-27 mm |
| PEDICEL | |
| Pubescence | Slight |
| Length | Average 133 mm; range 118-147 mm |
| Width | Average 3.5 mm; range 3-4 mm |
| Ratio (length/width) | 38 |
| Color | 5 GY 8/8 |
| FRUIT: | |
| Bearing | Day-neutral |
| Individual fruit weight | Seasonal average: 36 g |
| Shape | Broad conic |
| Achenes | Mostly flush |
| Glossiness | High gloss |
| External color evenness | Excellent external color uniformity |
| External color | 7.5 R 4/11 |
| Internal color | 7.5 R 5/13 |
| Band at top without achenes | Narrow to medium |
| Fruit Yield | Grams per plant: 2758 g |
| Firmness | 0.59 kg/force |
| Achene color | 7.5 Y 9/8 |

TABLE 2
Marketable yield of 'CBC019' compared to other cultivars known to
the Inventor in Watsonville, California during the 2024-2025 season.

| | Marketable yield (g/plant) | | | | | |
|---|---|---|---|---|---|---|
| Cultivar | April | May | June | July | August | Total |
| ‘CBC019’ | 142 | 485 | 870 | 986 | 275 | 2758 |
| ‘Cabrillo’ | 186 | 415 | 717 | 800 | 218 | 2336 |
| ‘Monterey’ | 195 | 485 | 648 | 600 | 178 | 2105 |

TABLE 3
Size of ‘CBC019’ compared to other cultivars known to the
Inventor in Watsonville, California during the 2024-2025season.
Fruit size (g/fruit)

| Cultivar | April | May | June | July | August | Average |
|---|---|---|---|---|---|---|
| ‘CBC019’ | 39.8 | 42.3 | 37.7 | 29.2 | 23.9 | 34.6 |
| ‘Cabrillo’ | 37.0 | 37.9 | 33.5 | 27.9 | 23.6 | 32.0 |
| ‘Monterey’ | 41.2 | 39.2 | 30.0 | 23.2 | 19.7 | 30.7 |

TABLE 4
Firmness of ‘CBC019’ compared to other cultivars known to the
inventor in Watsonville, CA during the 2024-2025season.
Firmness (kg/force)

| Cultivar | May | June | July | Average |
|---|---|---|---|---|
| ‘CBC019’ | 0.69 | 0.60 | 0.67 | 0.65 |
| ‘Cabrillo’ | 0.60 | 0.55 | 0.56 | 0.57 |
| ‘Monterey’ | 0.56 | 0.51 | 0.50 | 0.52 |

TABLE 5
DNA finger printing results of eight SSR markers.

| | Allelic Fingerprint Analysis | | | | | | | |
|---|---|---|---|---|---|---|---|---|
| Cultivar | M1 | M2 | M3 | M4 | M5 | M6 | M7 | M8 |
| ‘CBC019’ | 224, | 259, | 188, | 279, | 154, | 238, | 121, | 215, |
| | 229 | 264, | 190, | 289 | 156 | 257, | 124, | 222, |
| | | 271, | 216, | | | 264 | 130, | 228 |
| | | 275 | 232 | | | | 146 | |

I claim:

1. A new and distinct strawberry plant named ‘CBC019’ as described and illustrated herein.
